(19) United States
(12) Patent Application Publication
(10) Pub. No.: US 2011/0229538 A1
(43) Pub. Date: Sep. 22, 2011

(54) TOPICAL SKIN CARE COMPOSITION

(75) Inventors: Peter Matravers, Mission Viejo, CA (US); Deborah Hicks, Irvine, CA (US); Sheree Wiener, Laguna Niguel, CA (US); Michele Arth, Golden Valley, MN (US)

(73) Assignee: ARBONNE INTERNATIONAL LLC, Irvine, CA (US)

(21) Appl. No.: 13/050,532

(22) Filed: Mar. 17, 2011

Related U.S. Application Data
(60) Provisional application No. 61/314,958, filed on Mar. 17, 2010.

(51) Int. Cl. A61K 8/97; A61K 8/02; A61Q 19/00; A61P 29/00; A61Q 19/06; A61P 3/02; A61P 39/04; A61P 17/18; A61Q 17/04; A61P 17/00

(52) U.S. Cl. 424/401; 424/195.17; 424/59; 424/94.1; 424/702; 424/638; 424/642

(57) ABSTRACT

Topical skin care compositions containing a combination of ascorbic acid or a derivative thereof, brown algae extract, and a blend of botanical extracts comprising cucumber extract, watercress extract, birch leaf extract, red clover extract, and St. John's wort extract, and, optionally, ginseng extract, as well as methods of using the same, are disclosed.

This application claims the benefit of priority from U.S. Provisional Patent Application No. 61/314,958, which was filed on Mar. 17, 2010, the contents of which are incorporated herein in their entirety.

### BACKGROUND

1. Field

This disclosure relates to a topical skin care composition, as well as to methods of using the same.

2. Related Background Art

The skin, which is the largest (10 pounds) organ in the body, has two major cell types, namely, fibroblasts in the dermal layer and keratinocytes in the epidermis layer. The skin provides the body's first line of defense between the body's interior aggression and harmful environmental insults. Skin deteriorates with age as a natural consequence of prolonged exposure to internal and external factors. Internal deterioration factors include metabolic regenerative slowdown, free radicals, oxidative damage and loss of collagen. External factors include UV radiation, pollution, cigarette smoke, and environmental weathering.

Unfortunately conventional cosmetic products seldom augment the body's metabolism proactively. They only obscure and temporarily mask the signs of aging. It is therefore desirable to have a skin care method and composition which not only reduces the symptoms of deterioration but also treats the underlying causes.

### SUMMARY

The present disclosure is directed to a topical skin care composition comprising (a) an ascorbic acid source; (b) brown algae extract; (c) a blend of botanical extracts comprising cucumber extract, watercress extract, birch leaf extract, red clover extract, and St. John's wort extract; and (d) a cosmetically acceptable carrier. In a preferred embodiment, the blend of botanical extracts further comprises ginseng extract.

In a preferred embodiment of the present disclosure, the ascorbic acid source is ascorbic acid, tetrahexyldecyl ascorbate, ascorbyl palmitate, magnesium ascorbyl phosphate, or any combination thereof. In a preferred embodiment, the ascorbic acid source is present in an amount ranging from about 0.001% to about 40%, more preferably from about 0.1% to about 5%, and most preferably from about 0.5% to about 1%, by weight of the topical skin care composition.

Also in a preferred embodiment, the brown algae extract is obtained from a brown algae species selected from laminaria, ascophyllum, alaria, cladosiphan, durvillaea, ecklania, fucus, lessonia, macrocystis, sargassum, undoria, and combinations thereof. In a preferred embodiment, the brown algae extract is present in an amount ranging from about 0.001% to about 40%, more preferably from about 0.1% to about 10%, and most preferably from about 0.5% to about 5%, by weight of the topical skin care composition. In addition, the brown algae extract may contain a solvent in a most preferred amount ranging from about 90% to about 97% by weight of the brown algae extract.

Also in a preferred embodiment, the blend of botanical extracts is present in an amount ranging from about 0.001% to about 5%, more preferably from about 0.01% to about 2%, and most preferably from about 0.1% to about 1%, by weight of the topical skin care composition. In other embodiments, the blend of botanical extracts further comprises ginseng extract.

In a certain preferred embodiment, the ascorbic acid source is present in an amount ranging from about 0.5% to about 1% by weight of the topical skin care composition, the brown algae extract is present in an amount ranging from about 0.5% to about 5% by weight of the topical skin care composition, and the blend of botanical extracts is present in an amount ranging from about 0.1% to about 1% by weight of the topical skin care composition.

In a preferred embodiment, the cosmetically acceptable carrier is selected from purified water, oils, alcohols, glycols, and combinations thereof.

The topical skin care composition of the present disclosure may also further comprise additional ingredients such as penetration enhancers, humectants, lubricants, pharmaceutically active agents, color, fragrance, preservatives, antioxidants, chelators, neutralizers, amino acids, anti-inflammatory agents, anti-cellulite agents, anti-irritants, anti-tack agents, astringents, binders, catalysts, stabilizers, emollients, emulsifiers, surfactants, cell-signaling agents, essential oils, plant/botanical extracts, conditioners, film formers, gelling agents, foaming agents, exfoliants, vitamins, minerals, pH adjusters, proteins, peptides, neurotransmitters/inhibitors, tactile enhancers, saccharides, solvents or any combination thereof.

In certain embodiments, the topical skin care composition may be formulated as a cream, lotion, serum, facial cleanser, toner, eye cream, sunscreen, stick, spray, filled capsules, impregnated bandage, impregnated personal care device, impregnated towelette, gel, fluid/liquid, soap, transdermal patch, powder, liquid powder, cream powder, oil, butter, peel, scrub, mask, elixir, concentrate, capsule, semi-solid, or any other form that may be known in the art.

The present disclosure is also directed to a method of treating skin comprising the step of applying a topical skin care composition comprising (a) an ascorbic acid source; (b) brown algae extract; (c) a blend of botanical extracts comprising cucumber extract, watercress extract, birch leaf extract, red clover extract, and St. John's wort extract; and (d) a cosmetically acceptable carrier to the skin. In a preferred embodiment, the blend of botanical extracts further comprises ginseng extract. In a certain embodiment, the method further comprises an additional step of applying at least one additional skin care composition to the skin, wherein the at least one additional skin care composition comprises (a) an ascorbic acid source; (b) brown algae extract; and (c) a cosmetically acceptable carrier.

In a preferred embodiment, the topical skin care composition is applied to skin which suffers from a condition such as wrinkles, fine lines, hyperpigmentation, uneven tone, loss of firmness, creepiness (creepy skin texture), surface roughness, dark circles, under-eye puffiness, crow's feet, visible sun damage, redness, dryness, irritation, skin sagging, skin slackening, enlarged pores or any combination thereof. In a preferred embodiment, the topical skin care composition is topically applied in an amount and for a period of time sufficient to treat the skin for the condition treated. In preferred embodiments, the topical skin care composition is applied at least once a day or twice a day. In a certain embodiment, more than one topical skin care composition is applied.

In a preferred embodiment, the method of the present disclosure further comprises the step of: administering an oral supplement formulated to support skin health. In a certain embodiment, the oral supplement is administered at least once a day.

The present disclosure is also directed to a method of treating skin comprising the steps of: applying a topical skin care composition to the skin of the subject, and administering to a subject an oral supplement formulated to support skin health, wherein the topical skin care composition comprises (a) an ascorbic acid source; (b) brown algae extract; (c) a blend of botanical extracts comprising cucumber extract, watercress extract, birch leaf extract, red clover extract, and St. John's wort extract; and (d) a cosmetically acceptable carrier; and wherein the oral supplement comprises (a) bilberry extract, (b) quercetin, (c) beta-carotene, (d) co-enzyme Q-10, (e) lipoic acid, (f) vitamin A, (g) vitamin B, (h) vitamin C, (i) vitamin D, (j) vitamin E, (k) selenium, (l) zinc, and (m) copper. In a preferred embodiment, the blend of botanical extracts further comprises ginseng extract. In a certain embodiment, the method further comprises an additional step of applying at least one additional skin care composition to the skin of the subject, wherein the at least one additional skin care composition comprises (a) an ascorbic acid source; (b) brown algae extract; and (c) a cosmetically acceptable carrier.

### DETAILED DESCRIPTION

The present disclosure is directed to a topical skin care composition containing a combination of an ascorbic acid source, i.e., ascorbic acid and/or a derivative thereof, brown algae extract, and a blend of botanical extracts, as well as to a method of using the same. The skin care composition of the present disclosure effectively delivers nutrients to skin cells by formulating active ingredients in the most bioavailable form. The present combination of an ascorbic acid source, brown algae extract and a blend of botanical extracts of cucumber extract, watercress extract, birch leaf extract, red clover extract, St. John's wort extract, and optionally ginseng extract effectively increases cellular collagen production and inhibits glycation. Further the present combination promotes cellular rejuvenation and inhibits collagenase, tyrosinase, β-galactosidase and elastase activities. As a result, surprising performance benefits are achieved in alleviating fine lines and wrinkles, firming skin, evening skin tone, hydrating skin and promoting a younger appearance.

In a first embodiment, the present disclosure is directed to a topical skin care composition comprising (a) an ascorbic acid source; (b) brown algae extract; (c) a blend of botanical extracts comprising cucumber extract, watercress extract, birch leaf extract, red clover extract, and St. John's wort extract; and (d) a cosmetically acceptable carrier.

The ascorbic acid (vitamin C) source used in the present disclosure may be ascorbic acid, a derivative thereof, or some combination of ascorbic acid and a derivative thereof. In preferred embodiments of the disclosure, a stable, bioavailable form of ascorbic acid is used in the topical skin care composition. It is well known in the art that certain forms of vitamin C are more stable to formulation and more bioavailable for use upon application in skin care compositions. Any well known stable, bioavailable form of ascorbic acid may be used for purposes of this disclosure and can be purchased from known sources or made according to known synthetic procedures. Preferably the ascorbic acid derivative is an ester of ascorbic acid, a salt or a mixture thereof. Esters may be selected from fatty acid mono-, di-, tri-, or tetra-esters of ascorbic acid. Salts may be selected from phosphates and sulfates, with the cation being calcium, magnesium, sodium and the like. A preferred salt includes magnesium ascorbyl phosphate.

Suitable ascorbic acid derivatives include, without limitation, ascorbyl palmitate, ascorbyl laureate, ascorbyl myristate, ascorbyl stearate, ascorbyl dipalmitate, ascorbyl tripalmitate, ascorbyl tetrapalmitate, magnesium ascorbyl phosphate, and combinations thereof. Preferred ascorbic acid derivatives include ascorbyl tetrapalmitate, ascorbyl palmitate, magnesium ascorbyl palmitate, and combinations thereof. A preferred embodiment of the disclosure employs ascorbyl tetrapalmitate, which is also known as ascorbyl tetraisopalmitate, tetraisohexadecanoate L ascorbic acid, and tetrahexyldecyl ascorbate (all further references herein will use this last term).

According to the present disclosure, the ascorbic acid source is preferably present in an amount ranging from about 0.001% to about 40% by weight of the topical skin care composition, more preferably in an amount ranging from about 0.1% to about 5% by weight of the topical skin care composition, and most preferably in an amount ranging from about 0.5% to about 1%, by weight of the topical skin care composition.

The brown algae extract used in the present disclosure can be obtained from commercial sources or harvested according to known collection and extraction procedures. Preferably the brown algae extract is obtained from a brown algae species selected from laminaria, ascophyllum, alaria, cladosiphan, durvillaea, ecklania, fucus, lessonia, macrocystis, sargassum, undoria, and combinations thereof. In a particularly preferred embodiment of the disclosure, the brown algae extract is obtained from the species laminaria digitata such as the brown algae extract sold as Mitostime by Barnet Products Corporation (Englewood Cliffs, N.J.).

According to the present disclosure, the brown algae extract is preferably present in an amount ranging from about 0.001% to about 40% by weight of the topical skin care composition, more preferably in an amount ranging from about 0.1% to about 10% by weight of the topical skin care composition, and most preferably in an amount ranging from about 0.5% to about 5% by weight of the topical skin care composition. As one of ordinary skill in the art can readily appreciate, any brown algae extract will likely contain water, alcohol, glycol, glycerin, oil, or some other solvent. To that end, the concentration of active materials in the brown algae extract such as amino acids, polysaccharides (sugars), proteins, etc. may vary, and often are found in an amount ranging from about 0.5% to about 99%, preferably from about 1.5% to about 60%, and most preferably from about 3% to about 10%. The brown algae extract may contain water, or other solvent such as alcohol, glycol, glycerin or oil, in an amount ranging from about 1% to about 99.5%, preferably from about 40% to about 98.5%, and most preferably, from about 90% to about 97% by weight of the brown algae extract.

Generally it is desirable to include an amount of brown algae extract in the skin care composition sufficient to provide a daily topical dosage of (exposure to) brown algae active ingredients ranging preferably from about 0.05 grams to about 0.2 grams, more preferably about 0.079 grams. Likewise, it is desirable to include an amount of ascorbic acid and botanical extracts in the skin care composition sufficient to provide a daily topical dosage of (exposure to) ascorbic acid ranging preferably from about 0.011 grams to about 0.044 grams, and more preferably about 0.0275 grams, and a daily topical dosage of (exposure to) botanical extracts ranging preferably from about 0.08 grams to about 0.335 grams, and more preferably about 0.25 grams. This desirable daily topical dosage (exposure) amount takes into account that various products containing brown algae may be used in conjunction with one another, i.e., skin care regimen consisting of two or more skin care compositions, and that certain skin care compositions may be recommended for use more often than once daily. A day time routine may consist of a combination of two or more of the following products, which contain both brown algae extract and ascorbic acid or its derivatives: (1) facial cleanser, (2) facial toner, (3) facial serum, (4) eye crème, and (5) SPF 20 day cream. A night time routine may consist of a combination of two or more of the following products, which contain both the brown algae extract and ascorbic acid or its derivatives: (1) facial cleanser, (2) facial toner, (3) facial serum, (4) eye crème, and (5) night cream.

The blend of botanical extracts suitable for use in the present disclosure is a blend of cucumber (cucumis sativus) extract, watercress (nasturtium officinale) extract, birch leaf (betula alba leaf) extract, red clover (trifoleum pretense) extract, and St. John's wort (hypericum perforatum) extract. In a preferred embodiment, the blend of botanical extracts further comprises ginseng extract. Ginseng extract as used herein refers to the extract of ginseng root from the genus Panax, which includes Panax quinquefolius and Panax ginseng. Panax ginseng root extract is a preferred form for use in the present disclosure.

The botanical extracts suitable for use in the present disclosure can be obtained from commercial sources or harvested according to known collection and extraction procedures. As used herein, the term “extract” includes botanical actiphytes, as well as CO₂ botanical extracts, and aromatic botanical extracts. As one of ordinary skill in the art can readily appreciate, actiphytes are extracted botanicals using solvents such as glycerin, butylene glycol, propylene glycol, safflower oil, water and combinations thereof. In preferred embodiments, the botanical actiphytes are extracted using glycerin or butylene glycol. In addition, botanical extracts may contain water, alcohol, glycol, glycerin, oil, or some other solvent. The blend of botanical extracts may be supplied to the skin care composition of the present disclosure in a form which also comprises other ingredients such as preservatives, sodium pyroglutamic acid (sodium PCA), polyamino sugar condensate, etc.

Accordingly, as one of ordinary skill in the art can readily appreciate, any botanical extract will likely contain water, alcohol, glycol, glycerin, oil, or some other solvent. To that end, the concentration of active materials in a botanical extract may vary, and often is found in an amount ranging from about 10% to about 90%, preferably in an amount ranging from about 50% to about 80%, by weight of the extract.

In certain embodiments, additional botanical extracts known in the art as suitable for application to the skin may be added to the topical skin care composition. Preferred additional botanical extracts include, but are not limited to, fennel seed, green tea, rosemary, chamomile, orange, lemon, apple, sugar cane, alfalfa seed, Kudzu (Pueraria lobata) root, European or Common Beech (Fagus sylvatica), Saccharomyces cerevisiae, marine lavendar, beech bud, white lupine, and combinations thereof.

According to the present disclosure, the blend of botanical extracts is preferably present in an amount ranging from about 0.001% to about 5%, more preferably in an amount ranging from about 0.01% to about 2%, and most preferably in an amount ranging from about 0.1% to about 1%, by weight of the topical skin care composition.

In certain embodiments of the blend of botanical extracts, cucumber extract is present in an amount ranging from about 0.001% to about 2.0% by weight, more preferably from about 0.01% to about 0.2% by weight; watercress extract is present in an amount ranging from about 0.001% to about 2.0% by weight, more preferably from about 0.01% to about 0.2% by weight; birch leaf extract is present in an amount ranging from about 0.001% to about 2.0% by weight, more preferably from about 0.01% to about 0.2% by weight; red clover extract is present in an amount ranging from about 0.001% to about 2.0% by weight, more preferably from about 0.01% to about 0.2% by weight; and St. John's wort extract is present in an amount ranging from about 0.001% to about 2.0% by weight, more preferably from about 0.01% to about 0.2% by weight. Ginseng extract, when present, is preferably present in an amount ranging from about 0.001% to about 2.0%, and more preferably from about 0.01% to about 0.2%, by weight of the blend of botanical extracts. One of ordinary skill in the art will readily appreciate that the blend of botanical extracts can be included in the topical skin care composition by adding each extract individually, by adding some pre-combined combination of two or more extracts, or by combination thereof.

In a particularly preferred embodiment of the present disclosure, the ascorbic acid source is present in an amount ranging from about 0.5% to about 1% by weight of the topical skin care composition, the brown algae extract is present in an amount ranging from about 0.5% to about 5% by weight of the topical skin care composition, and the blend of botanical extracts is present in an amount ranging from about 0.1% to about 1% by weight of the topical skin care composition. In a further preferred embodiment, the ascorbic acid source comprises tetrahexyldecyl ascorbate, the brown algae extract is obtained from the laminaria digitata species and the blend of botanical extracts comprises cucumber extract, watercress extract, birch leaf extract, red clover extract, ginseng extract, and St. John's wort extract.

The cosmetically acceptable carrier used in the present disclosure can be any cosmetically acceptable carrier suitable for the particular form taken by the composition. The topical skin care composition of the present disclosure can be formulated as a cream, lotion, serum, facial cleanser, toner, eye cream, sunscreen, stick, spray, filled capsule, impregnated bandage, impregnated personal care device, impregnated towelette, gel, fluid/liquid, soap, transdermal patch, powder, liquid powder, cream powder, oil, butter, peel, scrub, mask, elixir, concentrate, capsule, semi-solid, or any other form known in the art. Preferably, the topical skin care composition of the present disclosure is formulated as a cream, toner, eye cream, sunscreen, or serum.

One of ordinary skill in the art will readily appreciate that, given the wide variety of forms in which the topical skin care composition can be formulated, the cosmetically acceptable carrier can be suitably chosen from the abundance of known cosmetically acceptable carriers in the art. Likewise the amount of cosmetically acceptable carrier used in the skin care composition of the present disclosure will vary given the type of formulation.

Suitable cosmetically acceptable carriers include, without limitation, purified water, oils, alcohols, glycols, and combinations thereof. The carrier may, in some embodiments, comprise capsules, encapsulates and delivery system vesicles, including but not limited to liposomes, niosomes, liquid crystals, vitaspheres, and q-somes, and combinations thereof. A preferred cosmetically acceptable carrier is water. According to the present disclosure, the cosmetically acceptable carrier is present in an amount ranging from preferably about 15% to about 99.99% by weight, and more preferably about 30% to about 98% by weight of the skin care composition.

Further the topical skin care composition of the present disclosure may also contain additional ingredients selected from penetration enhancers, humectants, lubricants, pharmaceutically active agents, color, fragrance, preservatives, antioxidants, chelators, neutralizers, amino acids, anti-inflammatory agents, anti-cellulite agents, anti-irritants, anti-tack agents, astringents, binders, catalysts, stabilizers, emollients, emulsifiers, surfactants, cell-signaling agents, essential oils, plant/botanical extracts, conditioners, film formers, gelling agents, foaming agents, exfoliants, vitamins, minerals, pH adjusters, proteins, peptides, neurotransmitters/inhibitors, tactile enhancers, saccharides, solvents, and combinations thereof. When present, these additional ingredients may be present in an amount ranging from about 0.001% to about 5% by weight of the topical skin care composition.

A preferred additional ingredient includes vitamin liposomes, i.e., liposome formulations containing one or more vitamins such as Vitaspheres (Croda/Sederma). Vitamin liposomes suitable for inclusion in the topical skin care compositions of the present invention include those formulated with mixtures of vitamins A and E, mixtures of vitamins C and E, etc. When present, the amount of vitamin liposomes ranges preferably from about 0.0001% to about 10%, more preferably from about 0.001% to about 5%, and most preferably from about 0.01% to about 1.5% by weight of the topical skin care composition.

The topical skin care compositions of the present disclosure can be readily made by one of ordinary skill in the art using conventional formulation techniques such as those set forth in the examples below.

A second embodiment of the present disclosure is directed to a method of treating skin comprising the step of applying the topical skin care composition of the first embodiment of the disclosure to the skin. Typically, the topical skin care composition is applied to skin which suffers from a condition selected from the group consisting of wrinkles, fine lines, hyperpigmentation, uneven tone, loss of firmness, creepiness (creepy skin texture), surface roughness, dark circles, under-eye puffiness, crow's feet, visible sun damage, redness, dryness, irritation, skin sagging, skin slackening, enlarged pores, and combinations thereof. Alternatively the topical skin care composition of the disclosure is applied to skin in order to prevent the effects of the conditions noted above.

In a preferred embodiment of the disclosure, the topical skin care composition is topically applied in an amount and for a period of time sufficient to treat or prevent one or more of the above-noted conditions. One of ordinary skill in the art can readily determine an appropriate amount for application, as well as an appropriate application frequency and/or duration. Preferably the topical skin care composition of the present disclosure is applied at least once a day, more preferably once or twice a day. In addition to these daily treatments, in another preferred embodiment, topical skin care compositions may be formulated for use on an every other day, weekly, monthly, etc. basis. Such compositions would likely take the form of a mask, exfoliant, skin refiner, skin booster elixir, supplemental face oil, cleaning wipe, concentrated treatment powder in combination with a serum, etc. The topical skin care composition of the present disclosure may be used as part of a skin care regimen, i.e., several products used regularly in conjunction with one another, one or more of which may be a topical skin care composition of the present disclosure; for example, a typical skin care regimen includes the use of a cleanser, toner, serum and eye cream twice daily, along with the use of a day cream once daily and a night cream once daily. In such a regimen, not all of the skin care compositions used may be topical skin care compositions of the present disclosure.

A targeted daily exposure via topical application to brown algae active materials preferably ranges from about 0.05 grams to about 0.2 grams, and more preferably is about 0.079 grams. A targeted daily exposure via topical application to ascorbic acid preferably ranges from about 0.011 grams to about 0.044 grams, and more preferably is about 0.0275 grams. A targeted daily exposure via topical application to botanical extracts preferably ranges from about 0.08 grams to about 0.335 grams, and more preferably is about 0.25 grams. These targeted daily exposure amounts represent the optimal daily dosage taking into account all products that may be used in conjunction and which contain varying amounts of brown algae, ascorbic acid and botanical extracts. For example, a skin care regimen may consist of two or more skin care compositions, and a certain skin care composition may be recommended for use more than once daily, but the targeted daily exposure values remain constant as the recommended total daily dosage.

In a preferred embodiment of the present disclosure, the method of treating skin further comprises the step of administering an oral supplement formulated to support skin health. As used herein, “support skin health” refers to an oral supplement which has a positive effect on the treatment and/or prevention of the skin conditions noted above or to an oral supplement which contributes to a positive effect, i.e., increases, speeds up, etc., on the treatment and/or prevention of the skin conditions noted above. Preferably the oral supplement comprises a combination of vitamins, minerals, and other nutrients. More preferably, the combination of vitamins, minerals and other nutrients are known to be useful in supporting skin health. In this embodiment of the disclosure, the oral supplement is administered at least once a day. While a desired dosage, i.e., daily dosage, of an oral supplement can be achieved in a singular dosage form, it is possible and sometimes even desirable to split a desired dosage, i.e., daily dosage, between two or more dosage forms.

An oral supplement preferred for use in the method of the present disclosure is that described in co-pending U.S. patent application Ser. No. ______ [filed concurrently herewith; entitled ORAL SUPPLEMENT; attorney docket no. 04072.000200.]. The entire disclosure of this co-pending application is incorporated by reference herein. In some cases, combined use of the topical skin care compositions of the present disclosure (especially in a regimen as set forth above) and the oral supplement of the co-pending application will be found to accelerate skin hydration and reduce fine lines in half the time as compared to the use of the topical skin care compositions of the instant disclosure only. In a preferred embodiment of the oral supplement of the co-pending application, the oral supplement comprises (a) bilberry extract, (b) quercetin, (c) beta-carotene, (d) co-enzyme Q-10, (e) lipoic acid, (f) vitamin A, (g) vitamin B, (h) vitamin C, (i) vitamin D, (j) vitamin E, (k) selenium, (l) zinc, and (m) copper.

In a certain embodiment, the method further comprises a step of applying at least one additional skin care composition to the skin, wherein the at least one additional skin care composition comprises (a) an ascorbic acid source; (b) brown algae extract; and (c) a cosmetically acceptable carrier. The additional skin care composition can be formulated as a cream, lotion, serum, facial cleanser, toner, eye cream, sunscreen, stick, spray, filled capsule, impregnated bandage, impregnated personal care device, impregnated towelette, gel, fluid/liquid, soap, transdermal patch, powder, liquid powder, cream powder, oil, butter, peel, scrub, mask, elixir, concentrate, capsule, semi-solid, or any other form known in the art. Preferably, the additional skin care composition is formulated as a facial cleanser, cream or facial mask. One of ordinary skill in the art will readily appreciate that, given the wide variety of forms in which a skin care composition can be formulated, the cosmetically acceptable carrier can be suitably chosen from the abundance of known cosmetically acceptable carriers in the art. Likewise the amount of cosmetically acceptable carrier used in the skin care composition of the present disclosure will vary given the type of formulation.

The at least one additional skin care composition may also contain additional ingredients selected from penetration enhancers, humectants, lubricants, pharmaceutically active agents, color, fragrance, preservatives, antioxidants, chelators, neutralizers, amino acids, anti-inflammatory agents, anti-cellulite agents, anti-irritants, anti-tack agents, astringents, binders, catalysts, stabilizers, emollients, emulsifiers, surfactants, cell-signaling agents, essential oils, plant/botanical extracts, conditioners, film formers, gelling agents, foaming agents, exfoliants, vitamins, minerals, pH adjusters, proteins, peptides, neurotransmitters/inhibitors, tactile enhancers, saccharides, solvents, and combinations thereof.

In a certain embodiment, the method of treating skin further comprises the step of administering an oral supplement formulated to support skin health and further comprises a step of applying at least one additional skin care composition to the skin, wherein the at least one additional skin care composition comprises (a) an ascorbic acid source; (b) brown algae extract; and (c) a cosmetically acceptable carrier.

Specific embodiments of the disclosure will now be demonstrated by reference to the following general methods of manufacture and examples. It should be understood that these examples are disclosed solely by way of illustration and should not be taken in any way to limit the scope of the present disclosure.

### Example 1

A topical SPF 20 day cream was prepared using the ingredients set forth in Table 1 below.

TABLE 1

| | Ingredient | % w/w |
|---|---|---|
| 1 | Deionized water | 55.8560 |
| 2 | Disodium EDTA | 0.1000 |
| 3 | Allantoin | 0.1000 |
| 4 | Panthenol | 0.1000 |
| 5 | Glycerin | 1.5000 |
| 6 | Phenoxyethanol | 1.0500 |
| | Ethylhexylglycerin | |
| 7 | Ethylhexylglycerin | 0.2500 |
| 8 | Glycerin | 0.1000 |
| | Water | |
| | Butylene glycol | |
| | Carbomer | |
| | Polysorbate 20 | |
| | Palmitoyl oligopeptide | |
| | Palmitoyl tetrapeptide-7 | |
| | (Matrixyl 3000, Croda/Sederma) | |
| 9 | Octocrylene | 8.0000 |
| 10 | Ethylhexyl salicylate (non-OTC) | 5.0000 |
| | Octisalate (OTC) | |
| 11 | PEG-40 stearate | 1.5000 |
| 12 | Glyceryl stearate SE | 2.2000 |
| 13 | Cetearyl alcohol | 3.0000 |
| | Ceteareth-20 | |
| 14 | Dimethicone | 1.0000 |
| 15 | Squalane | 0.1000 |
| 16 | Butyrospermum parkii (shea butter) | 0.1000 |
| 17 | Zinc oxide | 14.7000 |
| | C12-15 alkyl benzoate | |
| 18 | Polyacrylate-13 | 0.7000 |
| | Polyisobutene | |
| | Polysorbate 20 | |
| | (Sepiplus 400, Seppic) | |
| 19 | Deionized water | 2.0000 |
| 20 | Butylene glycol | 0.2000 |
| 21 | Magnesium ascorbyl phosphate | 0.1000 |
| | (Ronacare MAP/Ascorbyl PM, | |
| | Argan) | |
| 22 | Thioctic acid | 0.0001 |
| 23 | Water | 0.1000 |
| | Glycerin | |
| | Panthenol | |
| | Lecithin | |
| | stearamine | |
| | (Vitasphere S100/PA, Croda) | |
| 24 | Water | 0.2500 |
| | Borago officinalis seed oil | |
| | Tocopheryl acetate | |
| | Caprylic/capric triglyceride | |
| | Lecithin | |
| | Retinyl palmitate | |
| | (Vitasphere HAECL, | |
| | Croda/Sederma) | |
| 25 | Glycerin | 0.0625 |
| | Water | |
| | Cucumis sativus (cucumber) fruit | |
| | extract | |
| | (Active Organics) | |
| 26 | Butylene glycol | 0.0625 |
| | Water | |
| | Nasturtium officinale (watercress) | |
| | extract | |
| | (Active Organics) | |
| 27 | Butylene glycol | 0.0625 |
| | Water | |
| | Betula alba leaf (birch leaves) extract | |
| | (Active Organics) | |
| 28 | Glycerin | 0.0625 |
| | Water | |
| | Trifolium pratense (clover) flower | |
| | extract | |
| | (Active Organics) | |
| 29 | Glycerin | 0.0625 |
| | Water | |
| | Hypericum perforatum (St. John's | |
| | wort) extract | |
| | (Active Organics) | |
| 30 | Glycerin | 0.0625 |
| | Water | |
| | Chamomilla recutita (matricaria) | |
| | flower extract | |
| 31 | Butylene glycol | 0.0625 |
| | water | |
| | Rosmarinus officinalis (rosemary) | |
| | leaf extract | |
| 32 | Water | 0.5000 |
| | Laminaria digitata extract | |
| | (Mitostime, Barnet) | |
| 33 | Sodium PCA | 0.0063 |
| 34 | Polyamino sugar condensate | 0.0001 |
| | Urea | |
| 35 | Glycerin | 0.1000 |
| | Water | |
| | Pueraria lobata root extract | |
| 36 | Tocopherol | 0.2000 |
| 37 | Bisabolol | 0.2000 |
| 38 | Flavors & fragrances | 0.5500 |

The day cream was prepared by placing the water (80° C.) (1) in a sanitized jacketed stainless steel (ss) processing tank equipped with a mixer. Moderate agitation was begun and ingredients (2) through (8) were added in order mixing each material before adding the next. The mixture was stirred until all dissolved and mixture was homogenous. The water temperature was maintained at 80° C. for emulsification to occur. In a second jacketed ss tank equipped with mixer, ingredients (9) through (16) were combined and heated to 80° C. with moderate agitation until dissolved and mixture is homogenous. When oil phase in the second jacketed ss tank reached 80° C. and was homogenous, the mixture was set to fast homomixing, and ingredient (17) was added. The mixture was then mixed until the material was well-dispersed in the oil phase. This was maintained in the oil phase at 80° C. for emulsification. When both the oil phase mixture and the water phase mixture in the first jacketed ss tank were at temperature, the oil phase was added to the water phase with fast homomixing. This was mixed until homogenous. The mixture was then force cooled to 60° C. When the batch reached 60° C., it continued to be fast mixed and ingredient (18) was added. The batch was mixed until smooth. The batch was then force cooled to 40° C. In a separate container, ingredients (19) through (22) were combined at 35-40° C. water temperature and mixed until dissolved. This was then added to the main batch with moderate agitation and held at 40° C. This was mixed until the batch was homogenous. Moderate agitation continued and the ingredients (23) through (38) were added one at a time, mixing in each material before adding the next. The batch was mixed until homogenous.

The resulting topical SPF 20 day cream was an opaque, viscous emulsion, off-white to slightly yellow in color with an orange citrus scent. The pH at 25° C. was 7.80-8.90, the viscosity 4,000-10,000 cPs and the specific gravity at 25° C. 1.050-1.100.

### Example 2

A topical night cream was prepared using the ingredients set forth in Table 2 below.

TABLE 2

| | Ingredient | % w/w |
|---|---|---|
| 1 | Deionized water | 36.0189 |
| 2 | Pelargonium graveolens flower/leaf/stem | 10.0000 |
| | extract | |
| 3 | Lippia citriodora flower/leaf/stem extract | 10.0000 |
| 4 | Carbomer | 0.4000 |
| | (Carbopol Ultrez 10, Lubrizol/Noveon) | |
| 5 | Disodium EDTA | 0.0500 |
| 6 | Glycerin | 3.0000 |
| 7 | Chlorphenesin | 0.3000 |
| 8 | Ethylhexylglycerin | 1.0000 |
| 9 | Phenoxyethanol | 0.5000 |
| 10 | Potassium sorbate | 0.2500 |
| 11 | Glyceryl stearate | 3.0000 |
| | PEG-100 stearate | |
| 12 | Sorbitan stearate | 2.0000 |
| 13 | Myristyl myristate | 1.0000 |
| | Myristyl laurate | |
| 14 | Stearic acid | 2.5000 |
| 15 | Polysorbate 60 | 0.7000 |
| 16 | Cetyl alcohol | 2.5000 |
| 17 | Carthamus tinctorius (safflower) seed oil | 1.5000 |
| 18 | Helianthusannuus (sunflower) seed oil | 0.7000 |
| 19 | Isononyl isononanoate | 3.0000 |
| 20 | Dimethicone | 3.0000 |
| 21 | Hippophae rhamnoides oil | 0.5000 |
| 22 | Caprylic/capric triglyceride | 1.0000 |
| | Lavandula stoechas extract | |
| | (Lavandox, Barnet) | |
| 23 | Tetrahexyldecyl ascorbate | 0.5000 |
| | (BV-OSC, DD Chem Co/Barnet) | |
| 24 | Deionized water | 2.0000 |
| 25 | Tromethamine | 0.4000 |
| 26 | Spent grain wax | 1.0000 |
| | (Stimu-Tex, Thomas/Pentapharm) | |
| 27 | Butylene glycol | 0.5000 |
| 28 | Lecithin | 0.5000 |
| | Carnosine | |
| | Tocopherol | |
| | Silybum marianum/silybum marianum | |
| | fruit extract | |
| | Glycerin | |
| | Phenoxyethanol | |
| | Water | |
| | (Ameliox OA, Kemira/Mibelle) | |
| 29 | Aloe barbadensis leaf juice | 0.1000 |
| 30 | Sodium PCA | 0.1000 |
| 31 | Glycerin | 0.1000 |
| | Water | |
| | Cucumis sativus (cucumber) fruit extract | |
| | (Active Organics) | |
| 32 | Glycerin | 0.1000 |
| | Water | |
| | Panax ginseng root extract | |
| | (Active Organics) | |
| 33 | Butylene glycol | 0.1000 |
| | Water | |
| | Betula alba leaf (birch leaves) extract | |
| | (Active Organics) | |
| 34 | Glycerin | 0.1000 |
| | Water | |
| | Trifolium pratense (clover) flower extract | |
| | (Active Organics) | |
| 35 | Glycerin | 0.1000 |
| | Water | |
| | Hypericum perforatum (St. John's wort) | |
| | extract | |
| | (Active Organics) | |
| 36 | Butylene glycol | 0.1000 |
| | Water | |
| | Nasturtium officinale (watercress) extract | |
| | (Active Organics) | |
| 37 | Magnesium ascorbyl phosphate | 0.2000 |
| | (Ronacare MAP/Ascorbyl PM, Argan) | |
| 38 | Glycine soja (soybean) protein | 1.0000 |
| | (Elhibin, Thomas/Pentapharm) | |
| 39 | Polyamino sugar condensate | 0.1000 |
| | Urea | |
| 40 | Thioctic acid | 0.0001 |
| 41 | Sodium riboflavin phosphate | 0.001 |
| 42 | Water | 1.0000 |
| | Fagus sylvatica bud extract | |
| | (Gatuline RC BIO, Lipscomb/Gatefosse) | |
| 43 | Water | 1.0000 |
| | Laminaria digitata extract | |
| | (Mitostime, Barnet) | |
| 44 | Glycerin | 1.0000 |
| | Water | |
| | Pueraria lobata root extract | |
| 45 | Tocopherol | 0.1000 |
| 46 | Retinyl palmitate | 0.1000 |
| 47 | Corn starch modified | 1.5000 |
| 48 | Glycerin | 3.0000 |
| | Water | |
| | Butylene | |
| | Glycol | |
| | Carbomer | |
| | Polysorbate 20 | |
| | Palmitoryl oligopeptide | |
| | Palmitoyl tetrapeptide-7 | |
| | (Matrixyl 3000, Croda/Sederma) | |
| 49 | Flavors & fragrances | 0.0300 |
| 50 | Water | 0.2500 |
| | Borago officinalis seed oil | |
| | Tocopheryl acetate | |
| | Caprylic/capric triglyceride | |
| | Lecithin | |
| | Retinyl palmitate | |
| | (Vitasphere HAECL, Croda/Sederma) | |
| 51 | Water | 0.1000 |
| | Glycerin | |
| | Panthenol | |
| | Lecithin | |
| | Stearamine | |
| | (Vitasphere S100/PA, Croda) | |
| 52 | Sodium hyaluronate | 1.0000 |
| | (Sodium Hyaluronate 1% non-animal | |
| | derived, American Intl Chem) | |
| 53 | Water | 0.5000 |
| | hydrogenated lecithin | |
| | tocopheryl acetate | |
| | retinyl palmitate | |
| | (liposomes vitamin A & E, | |
| | Collaborative) | |
| 54 | Water | 0.5000 |
| | Hydrogenated lecithin | |
| | tocopheryl acetate | |
| | ascorbyl palmitate | |
| | (liposomes vitamins C & E, | |
| | Collaborative) | |

The sample was prepared by placing the ingredients (1) through (4) including water at 35-40° C. in a sanitized, jacketed stainless steel (ss) processing tank equipped with a mixer. The mixture was mixed at a moderate speed until the solids were dissolved and the mixture was homogenous. The mixture was heated to 80° C., and while continuing to mix, ingredients (5) through (10) were added. The mixing continued until the solids were dissolved and the mixture was homogenous. The mixture was maintained at 80° C. for emulsification. In a separate jacketed ss tank equipped with a mixer, ingredients (11) through (22) were combined and heated to 80° C. with moderate agitation, until all solids were dissolved and the mixture was homogenous. The oil phase was then added to the water phase with fast homomixing and then force cooled to 75° C. When the batch reached 75° C., under moderate mixing, ingredient (23) was added and the batch mixed until homogenous. In separate container, ingredients (24) through (25) were combined (water at 80° C.) and mixed until dissolved. This was then added to the main batch with moderate agitation and held at 75° C. This was mixed until batch was homogenous, then force cooled to 70° C. Moderate agitation continued, and ingredient (26) was added. The batch was mixed until homogenous then force cooled to 40° C. In a separate container, ingredients (27) through (44) were added and mixed until homogenous and all solids dissolved. It was then added to the main batch with moderate agitation at 35° C. and mixed until homogenous. While continuing to mix with moderate agitation, ingredients (45) through (54) were added in order, mixing in each one before adding the next. This was then mixed until homogenous.

The resulting topical night cream was an opaque, viscous emulsion, light yellow to ivory in color with an orange citrus scent. The pH at 25° C. was 5.20-6.20, the viscosity 22,000-52,000 cPs and the specific gravity at 25° C. 0.975-1.025.

### Example 3

A topical eye cream was prepared using the ingredients set forth in Table 3 below.

TABLE 3

| | Ingredient | % w/w |
|---|---|---|
| 1 | Deionized water | 51.7448 |
| 2 | Carbomer | 0.1000 |
| | (Carbopol Ultrez 10, Lubrizol/Noveon) | |
| 3 | Allantoin | 0.1000 |
| 4 | Potassium sorbate | 0.3000 |
| 5 | Disodium EDTA | 0.1000 |
| 6 | Phenoxyethanol | 1.0500 |
| | Ethylhexylglycerin | |
| 7 | Glycerin | 2.0000 |
| 8 | Xanthan gum | 0.1000 |
| 9 | Cetyl alcohol | 0.7000 |
| 10 | Glyceryl stearate | 3.2000 |
| | PEG-100 stearate | |
| 11 | Sorbitan stearate | 0.8000 |
| 12 | Stearic acid | 1.6000 |
| 13 | Cetearyl alcohol | 0.8000 |
| | Ceteareth-20 | |
| 14 | Camellia oil | 1.6000 |
| 15 | Sesamum indicum (sesame) seed oil | 3.2000 |
| | (Sesame oil refined, Arista) | |
| 16 | Decyl oleate | 1.6000 |
| 17 | Squalane | 2.4000 |
| 18 | Octyldodecyl neopentanoate | 3.2000 |
| 19 | Glyceryl stearate | 1.6000 |
| 20 | Tetrahexyldecyl ascorbate | 0.5000 |
| | (BV-OSC, DD Chem Co/Barnet) | |
| 21 | Deionized water | 0.5000 |
| 22 | Tromethamine | 0.1200 |
| 23 | Caprylic/capric triglyceride | 1.0000 |
| | Lavandula stoechas extract | |
| | (Lavandox, Barnet) | |
| 24 | Butylene glycol | 1.0000 |
| 25 | Glycerin | 0.0500 |
| | Water | |
| | Camellia oleifera leaf extract | |
| 26 | Glycerin | 0.0500 |
| | Water | |
| | Cucumis sativus (cucumber) fruit extract | |
| | (Active Organics) | |
| 27 | Glycerin | 0.0500 |
| | Water | |
| | Trifolium pratense (clover) flower extract | |
| | (Active Organics) | |
| 28 | Glycerin | 0.0500 |
| | Water | |
| | Panax ginseng root extract | |
| | (Active Organics) | |
| 29 | Glycerin | 0.0100 |
| | Rosmarinus officinalis (rosemary) leaf extract | |
| 30 | Butylene glycol | 0.0500 |
| | Water | |
| | Nasturtium officinale (watercress) extract | |
| | (Active Organics) | |
| 31 | Butylene glycol | 0.0500 |
| | Water | |
| | Betula alba leaf (birch leaves) extract | |
| | (Active Organics) | |
| 32 | Glycerin | 0.0500 |
| | Water | |
| | Hypericum perforatum (St. John's wort) | |
| | extract | |
| | (Active Organics) | |
| 33 | Sodium PCA | 0.0050 |
| 34 | Polyamino sugar condensate | 0.0001 |
| | Urea | |
| 35 | Water | 0.0100 |
| | Glycerin | |
| | Carnitine | |
| | Caffeine | |
| | Coenzyme A | |
| | (Coaxel, Croda/Sederma) | |
| 36 | Water | 5.0000 |
| | Medicago sativa (alfalfa) seed extract | |
| | Hydrolyzed lupine protein | |
| | (Eye Regener, Silab) | |
| 37 | Deionized water | 0.5000 |
| 38 | Copper gluconate | 0.0100 |
| 39 | Deionized water | 2.0000 |
| 40 | Butylene glycol | 1.0000 |
| 41 | Magnesium ascorbyl phosphate | 0.2000 |
| | (Ronacare MAP/Ascorbyl PM, Argan) | |
| 42 | Thioctic acid | 0.0001 |
| 43 | Flavors & fragrances | 0.2500 |
| 44 | Glycerin | 3.0000 |
| | Water | |
| | Butylene glycol | |
| | Carbomer | |
| | Polysorbate 20 | |
| | Palmitoyl oligopeptide | |
| | Palmitoyl tetrapeptide-7 | |
| | (Matrixyl 3000, Croda/Sederma) | |
| 45 | Water | 0.2500 |
| | Borago officinalis seed oil | |
| | Tocopheryl acetate | |
| | Caprylic/capric triglyceride | |
| | Lecithin | |
| | Retinyl palmitate | |
| | (Vitasphere HAECL, Croda/Sederma) | |
| 46 | Water | 0.1000 |
| | Glycerin | |
| | Panthenol | |
| | Lecithin | |
| | Stearamine | |
| | (Vitasphere S100/PA, Croda) | |
| 47 | Water | 0.5000 |
| | hydrogenated lecithin | |
| | tocopheryl acetate | |
| | retinyl palmitate | |
| | (liposomes vitamin A & E, Collaborative) | |
| 48 | Water | 0.5000 |
| | Hydrogenated lecithin | |
| | tocopheryl acetate | |
| | ascorbyl palmitate | |
| | (liposomes vitamins C & E, Collaborative) | |
| 49 | Water | 3.0000 |
| | Laminaria digitata extract | |
| | (Mitostime, Barnet) | |
| 50 | Saccharomyces cerevisiae extract | 4.0000 |
| | (Actiflow EL, Silab) | |

The noted amount of ingredient (1) (deionized water at 35-40° C.) was metered into a clean, jacketed ss processing tank equipped with a mixer. Fast propeller mixing was begun and ingredient (2) was slowly sifted into the vortex and mixed until smooth. Heating to 75-80° C. with moderate agitation was begun, and ingredients (3) through (6) were added. Ingredients (7) and (8) were premixed and added to the processing tank at 75-80° C. with moderate agitation. The water phase was held at 80° C. for emulsification. In a dry jacketed ss auxiliary mixing tank, ingredients (9) through (20) were combined. This mixture was heated to 80° C. while mixing all ingredients until completely dissolved and homogenized. This oil phase was held at 80° C. for emulsification. When the oil phase and water phase were both at temperature, the oil phase was transferred into the water phase with fast homomixing. Ingredients (21) and (22) were premixed (water 35-40° C.) and then added to the processing tank with homomixing until a smooth, homogenous emulsion was obtained. Cooling to 65° C. was begun with moderate agitation. When the batch reached 65° C., ingredients (23) was added and mixed until uniform. Then cooling was continued to 40° C. When the batch temperature reached 40° C., ingredients (24) through (36) were added in order, mixing in each material before adding next. Mixing was continued until uniform. Ingredients (37) and (38) were premixed (water at 35-40° C.) and added to the processing tank with moderate agitation. Ingredient (39—water at 35-40° C.) was dispersed with ingredients (40) through (42) and added to the processing tank with moderate agitation. Moderate agitation was continued and ingredients (43) through (50) were added and mixed until uniform. The batch was cooled with slow mixing to 35° C.

The resulting topical eye cream was an opaque, viscous emulsion, off white in color with an orange citrus scent. The pH at 25° C. was 5.00-6.00, the viscosity 9,000-22,000 cPs and the specific gravity at 25° C. 0.920-1.005.

### Example 4

A topical facial serum was prepared using the ingredients set forth in Table 4 below.

TABLE 4

| | Ingredient | % w/w |
|---|---|---|
| 1 | Deionized water | 34.2250 |
| 2 | Magnesium aluminum silicate | 0.2000 |
| 3 | Deionized water | 30.0000 |
| 4 | Carbomer | 0.4500 |
| | (Carbopol Ultrez 10, Lubrizol/Noveon) | |
| 5 | Butylene glycol | 1.0000 |
| 6 | Polymethyl methacrylate | 3.0000 |
| 7 | Salicylic acid | 0.1000 |
| | (Vivion/Rhodia) | |
| 8 | Tetrasodium EDTA | 0.1000 |
| 9 | Potassium sorbate | 0.3000 |
| 10 | Phenoxyethanol | 0.9500 |
| 11 | Allantoin | 0.1000 |
| 12 | Panthenol | 0.1000 |
| 13 | Polysorbate 40 | 1.4000 |
| 14 | Cyclopentasiloxane | 4.9000 |
| | Acrylate/dimethicone copolymer | |
| | (KP 545, Chemtec/ShinEtsu) | |
| 15 | Cyclopentasiloxane | 2.1000 |
| | Dimethiconol | |
| 16 | Silica | 0.7000 |
| | Titanium dioxide | |
| | Iron oxides | |
| 17 | Tetrahexyldecyl ascorbate | 0.5000 |
| | (BV-OSC, DD Chem Co/Barnet) | |
| 18 | Caprylic/capric triglyceride | 1.0000 |
| | Lavandula stoechas extract | |
| | (Lavandox, Barnet) | |
| 19 | Hippophae rhamnoides oil | 0.1000 |
| 20 | Flavors & fragrances | 0.1250 |
| 21 | Deionized water | 0.9000 |
| 22 | Sodium hydroxide | 0.4500 |
| 23 | Polyacrylate-13 | 2.1000 |
| | Polyisobutene | |
| | Polysorbate 20 | |
| | (Sepiplus 400, Seppic) | |
| 24 | Saccharum officinarum (sugar cane) extract | 2.0000 |
| | Citrus medica limonum (lemon) extract | |
| | Citrus aurantium dulcis (orange) fruit extract | |
| | Pyrus malus (apple) fruit extract | |
| | Camellia sinensis leaf extract | |
| | (MFA Complex, Barnet) | |
| 25 | Glycerin | 0.1250 |
| | Water | |
| | Cucumis sativus (cucumber) fruit extract | |
| | (Active Organics) | |
| 26 | Butylene glycol | 0.1250 |
| | Water | |
| | Nasturtium officinale (watercress) extract | |
| | (Active Organics) | |
| 27 | Butylene glycol | 0.1250 |
| | Water | |
| | Betula alba leaf (birch leaves) extract | |
| | (Active Organics) | |
| 28 | Glycerin | 0.1250 |
| | Water | |
| | Trifolium pratense (clover) flower extract | |
| | (Active Organics) | |
| 29 | Glycerin | 0.1250 |
| | Water | |
| | Panax ginseng root extract | |
| | (Active Organics) | |
| 30 | Glycerin | 0.1250 |
| | Water | |
| | Hypericum perforatum (St. John's wort) | |
| | extract | |
| | (Active Organics) | |
| 31 | Sodium PCA | 0.1250 |
| 32 | Aloe barbadensis leaf juice | 0.2000 |
| 33 | Sodium hyaluronate | 1.0000 |
| | (Sodium hyaluronate 1% non-animal derived, | |
| | American Intl Chem) | |
| 34 | Water | 1.0000 |
| | Fagus sylvatica bud extract | |
| | (Gatuline RC BIO, Lipscomb/Gattefosse) | |
| 35 | Glycerin | 1.0000 |
| | Water | |
| | Pueraria lobata root extract | |
| | (Phytessence Kudzu, Croda) | |
| 36 | Water | 0.5000 |
| | Hydrogenated lecithin | |
| | tocopheryl acetate | |
| | ascorbyl palmitate | |
| | (liposomes vitamins C & E, Collaborative) | |
| 37 | Water | 0.5000 |
| | hydrogenated lecithin | |
| | tocopheryl acetate | |
| | retinyl palmitate | |
| | (liposomes vitamin A & E, Collaborative) | |
| 38 | Water | 0.1000 |
| | Glycerin | |
| | Panthenol | |
| | Lecithin | |
| | Stearamine | |
| | (Vitasphere S100/PA, Croda) | |
| 39 | Water | 0.2500 |
| | Borago officinalis seed oil | |
| | Tocopheryl acetate | |
| | Caprylic/capric triglyceride | |
| | Lecithin | |
| | Retinyl palmitate | |
| | (Vitasphere HAECL, Croda/Sederma) | |
| 40 | Lactic acid | 0.1250 |
| 41 | Sodium lactate | 0.3400 |
| 42 | Deionized water | 2.0000 |
| 43 | Magnesium ascorbyl phosphate | 0.2000 |
| | (Ronacare MAP/Ascorbyl PM, Argan) | |
| 44 | Thioctic acid | 0.0100 |
| 45 | Polyamino sugar condensate | 0.1000 |
| | Urea | |
| 46 | Water | 5.0000 |
| | Laminaria digitata extract | |
| | (Mitostime, Barnet) | |

Ingredient (1—water at 80° C.) was metered into a sanitized, jacketed ss processing tank equipped with a mixer. Fast homomixing was begun, and ingredient (2) was slowly sifted into the vortex. Homomixing was continued for 30 minutes. In a separate ss auxiliary processing tank, ingredients (3) and (4) (water at 35-40° C.) were premixed until dispersed. This was added to the batch with fast mixing at 80° C. and mixed until homogenous. Ingredients (5) through (12) were added to the batch at 80° C. with moderate agitation one at a time, making sure each material was dissolved and the batch was homogenous before adding next material. Then the batch was force cooled to 65° C. In a separate container, ingredients (13) through (20) were combined and heated to 65° C. and mixed until all materials were completely dissolved and homogeneous. The oil phase was added to the batch at 65° C. and homomixed until the batch was smooth. In a separate container, ingredients (21) and (22) (water at 80° C.) were premixed until solids were dissolved. Then this was added to the batch at 65° C. with moderate agitation and mixed until the batch is smooth and homogenous. Ingredient (23) was added to the batch at 65° C. with moderate agitation and mixed until smooth and lump-free. The batch was force cooled to 40° C. When batch temperature reached 40° C., ingredients (24) through (41) were added in order, mixing in each material before adding the next. The batch was mixed until uniform. Ingredients (42) through (45) (water at 35-40° C.) were premixed and mixed until dissolved. This was then added to the main processing tank with moderate agitation and cooling was continued to 35° C. When the batch temperature reached 35° C., ingredient (46) was added and mixed until completely uniform.

The resulting facial serum was an opaque, shiny, semi-viscous emulsion, ivory to light beige in color with an orange citrus scent. The pH at 25° C. was 4.70-5.70, the viscosity 3,000-5,000 cPs and the specific gravity at 25° C. 1.008-1.058.

### Example 5

A facial cleanser was prepared using the ingredients set forth in Table 5.

TABLE 5

| | Ingredient | % w/w |
|---|---|---|
| 1 | Deionized water | 40.2999 |
| 2 | Disodium EDTA | 0.1000 |
| 3 | Sodium cocoyl isethionate, | 27.0000 |
| | stearic acid | |
| 4 | Glycol stearate | 1.5000 |
| 5 | PEG-80 sorbitan laurate | 8.0000 |
| 6 | Sodium lauroamphoacetate | 5.0000 |
| 7 | Disodium lauroamphodiacetate | 5.0000 |
| | sodium trideceth sulfate | |
| | hexylene glycol | |
| | (Miracare 2MHT, Chemtec/Rhodia) | |
| 8 | Phenoxyethanol | 0.9500 |
| 9 | Glyceryl undecylenate | 0.3000 |
| 10 | Cocamidopropyl betaine | 6.0000 |
| 11 | Sodium C14-16 olefin sulfonate | 3.0000 |
| 12 | Polysorbate 20 | 0.8000 |
| 13 | Flavor and fragrance | 0.8000 |
| 14 | Hippophae rhamnoides oil | 0.0100 |
| 15 | Deionized water | 0.5000 |
| 16 | Butylene glycol | 0.1000 |
| 17 | Thioctic acid | 0.0001 |
| 18 | Magnesium ascorbyl phosphate | 0.0500 |
| | (Ronacare MAP/Ascorbyl PM, Argan) | |
| 19 | Water | 0.0200 |
| | Glycerin | |
| | Panthenol | |
| | Lecithin | |
| | Stearamine | |
| | (Vitasphere S100/PA, Croda) | |
| 20 | Water | 0.0100 |
| | hydrogenated lecithin | |
| | tocopheryl acetate | |
| | retinyl palmitate | |
| | (liposomes vitamin A & E, Collaborative) | |
| 21 | Water | 0.0100 |
| | Hydrogenated lecithin | |
| | tocopheryl acetate | |
| | ascorbyl palmitate | |
| | (liposomes vitamins C & E, Collaborative) | |
| 22 | Water | 0.0500 |
| | Borago officinalis seed oil | |
| | Tocopheryl acetate | |
| | Caprylic/capric triglyceride | |
| | Lecithin | |
| | Retinyl palmitate | |
| | (Vitasphere HAECL, Croda/Sederma) | |
| 23 | Water | 0.5000 |
| | Laminaria digitata extract | |
| | (Mitostime, Barnet) | |

The noted amount ingredient (1) (water at 80° C.) was metered into a sanitized, jacketed ss processing tank equipped with a mixer. Ingredients (2) through (4) were added with moderate agitation and mixed until uniform and all solids dissolved for a minimum of 30 minutes. Moderate agitation was continued, and ingredients (5) through (9) were added at 80° C. and mixed until uniform and all solids were dissolved and the batch was homogenous. Moderate agitation was continued, and ingredients (10) and (11) were added at 80° C. and mixed for a minimum of 30 minutes. The batch was force cooled to 40° C. In a separate container, ingredients (12) through (14) were premixed until homogenous and added to the batch at 40° C. with moderate agitation. In a separate container, ingredients (15) through (18) (water at 35-40° C.) were premixed until all solids were dissolved. This was added to the batch at 40° C. with moderate agitation. Ingredients (19) through (23) were added to the batch at 40° C. with moderate agitation and each mixed until dissolved before adding the next material. The batch was mixed until uniform.

The resulting facial cleanser was an opaque, shiny, viscous emulsion, white to off-white in color with an orange citrus scent. The pH at 25° C. was 5.70-6.70, the viscosity 10,000-30,000 cPs and the specific gravity at 25° C. 0.990-1.060.

### Example 6

A facial toner was prepared using the ingredients set forth in Table 6 below.

TABLE 6

| | Ingredient | % w/w |
|---|---|---|
| 1 | Deionized water | 60.9864 |
| 2 | Disodium EDTA | 0.1000 |
| 3 | Potassium sorbate | 0.3000 |
| 4 | Glycerin | 3.0000 |
| 5 | Thioctic acid | 0.0001 |
| 6 | Magnesium ascorbyl phosphate | 0.0100 |
| | (Ronacare MAP/Ascorbyl PM, Argan) | |
| 7 | Hamamelis virginiana (witch hazel) water | 30.0000 |
| 8 | Butylene glycol | 0.6000 |
| 9 | Glycerin | 0.0300 |
| | Water | |
| | Cucumis sativus (cucumber) fruit extract | |
| | (Active Organics) | |
| 10 | Butylene glycol | 0.0300 |
| | Water | |
| | Nasturtium officinale (watercress) extract | |
| | (Active Organics) | |
| 11 | Butylene glycol | 0.0300 |
| | Water | |
| | Betula alba leaf (birch leaves) extract | |
| | (Active Organics) | |
| 12 | Glycerin | 0.0300 |
| | Water | |
| | Trifolium pratense (clover) flower extract | |
| | (Active Organics) | |
| 13 | Glycerin | 0.0300 |
| | Water | |
| | Panax ginseng root extract | |
| | (Active Organics) | |
| 14 | Glycerin | 0.0300 |
| | Water | |
| | Hypericum perforatum (St. John's wort) | |
| | extract | |
| | (Active Organics) | |
| 15 | Sodium PCA | 0.0030 |
| 16 | Polyamino sugar condensate | 0.0001 |
| | Urea | |
| 17 | Water | 0.0100 |
| | hydrogenated lecithin | |
| | tocopheryl acetate | |
| | retinyl palmitate | |
| | (liposomes vitamin A & E, Collaborative) | |
| 18 | Water | 0.0100 |
| | Hydrogenated lecithin | |
| | tocopheryl acetate | |
| | ascorbyl palmitate | |
| | (liposomes vitamins C & E, Collaborative) | |
| 19 | Water | 0.1000 |
| | Glycerin | |
| | Panthenol | |
| | Lecithin | |
| | Stearamine | |
| | (Vitasphere S100/PA, Croda) | |
| 20 | Water | 0.0100 |
| | Borago officinalis seed oil | |
| | Tocopheryl acetate | |
| | Caprylic/capric triglyceride | |
| | Lecithin | |
| | Retinyl palmitate | |
| | (Vitasphere HAECL, Croda/Sederma) | |
| 21 | Water | 0.5000 |
| | Laminaria digitata extract | |
| | (Mitostime, Barnet) | |
| 22 | Phenoxyethanol | 0.9500 |
| 23 | Polysorbate 80 | 1.5000 |
| 24 | Isoceteth-20 | 1.5000 |
| 25 | Flavor & fragrance | 0.1400 |
| 26 | Hippophae rhamnoides oil | 0.1000 |
| 27 | Deionized water | 0.0001 |
| 28 | Sodium hydroxide | 0.0001 |
| 29 | Deionized water | 0.0001 |
| 30 | Citric acid | 0.0001 |

The noted amount ingredient (1) (water at 80° C.) was metered into a sanitized, jacketed ss processing tank equipped with a mixer. Ingredients (2) and (3) were added with moderate agitation and mixed until uniform and all solids dissolved. In a sanitized, jacketed ss auxiliary mixing vessel, ingredients (4) and (5) were combined and then added to the processing tank at 80° C. with moderate agitation. The batch was force cooled to 40° C. with moderate agitation. When the batch temperature reached 40° C., ingredients (6) through (21) were added in order, mixing in each material before adding the next. In an auxiliary ss mixing tank, ingredients (22) through (26) were premixed until a clear solution was obtained. This was added to the batch at 40° C. with moderate agitation. Ingredients (27) and (28) were premixed (water at 35-40° C.) and added in increments only as needed for pH adjustment. Ingredients (29) and (30) were premixed (water at 80° C.) and added in increments only as needed for pH adjustment. The batch was mixed until completely homogenous and filtered through a filter press.

The resulting facial toner was a clear, thin liquid, slightly yellow to yellow in color with an orange citrus scent. The pH at 25° C. was 4.75-5.75 and the specific gravity at 25° C. 0.973-1.023.

### Example 7

The oral supplement described in co-pending U.S. patent application Ser. No. ______ [filed concurrently herewith; entitled ORAL SUPPLEMENT; attorney docket no. 04072.000200.] was administered twice daily, i.e., in the morning (a.m.) and in the evening (p.m.), to a female subject. In the morning, the female subject also used a cleanser, toner, serum, eye cream and day cream such as those set forth in the examples above. In the evening, the female subject also used a cleanser, toner, serum, eye cream and night cream such as those set forth in the examples above.

While the disclosure has been described above with reference to specific embodiments thereof, it is apparent that many changes, modifications, and variations can be made without departing from the concept disclosed herein. Accordingly, it is intended to embrace all such changes, modifications, and variations that fall within the spirit and broad scope of the appended claims.

What is claimed is:

1. A topical skin care composition comprising:
(a) an ascorbic acid source;
(b) brown algae extract;
(c) a blend of botanical extracts comprising cucumber extract, watercress extract, birch leaf extract, red clover blossom extract, and St. John's wort extract; and
(d) a cosmetically acceptable carrier.

2. The topical skin care composition according to claim 1, wherein the ascorbic acid source is selected from the group consisting of ascorbic acid, tetrahexyldecyl ascorbate, ascorbyl palmitate, magnesium ascorbyl phosphate, and combinations thereof.

3. The topical skin care composition according to claim 1, wherein the ascorbic acid source is present in an amount ranging from about 0.001% to about 40% by weight of the topical skin care composition.

4. The topical skin care composition according to claim 3, wherein the ascorbic acid source is present in an amount ranging from about 0.1% to about 5% by weight of the topical skin care composition.

5. The topical skin care composition according to claim 4, wherein the ascorbic acid source is present in an amount ranging from about 0.5% to about 1% by weight of the topical skin care composition.

6. The topical skin care composition according to claim 1, wherein the brown algae extract is obtained from a brown algae species selected from the group consisting of laminaria, ascophyllum, alaria, cladosiphan, durvillaea, ecklania, fucus, lessonia, macrocystis, sargassum, undoria, and combinations thereof.

7. The topical skin care composition according to claim 1, wherein the brown algae extract is present in an amount ranging from about 0.001% to about 40% by weight of the topical skin care composition.

8. The topical skin care composition according to claim 7, wherein the brown algae extract is present in an amount ranging from about 0.1% to about 10% by weight of the topical skin care composition.

9. The topical skin care composition according to claim 8, wherein the brown algae extract is present in an amount ranging from about 0.5% to about 5% by weight of the topical skin care composition.

10. The topical skin care composition according to claim 1, wherein the brown algae extract contains a solvent in an amount ranging from about 90% to about 97% by weight of the brown algae extract.

11. The topical skin care composition according to claim 1, wherein the blend of botanical extracts is present in an amount ranging from about 0.001% to about 5% by weight of the topical skin care composition.

12. The topical skin care composition according to claim 11, wherein the blend of botanical extracts is present in an amount ranging from about 0.01% to about 2% by weight of the topical skin care composition.

13. The topical skin care composition according to claim 12, wherein the blend of botanical extracts is present in an amount ranging from about 0.1% to about 1% by weight of the topical skin care composition.

14. The topical skin care composition according to claim 1, wherein the ascorbic acid source is present in an amount ranging from about 0.5% to about 1% by weight of the topical skin care composition, wherein the brown algae extract is present in an amount ranging from about 0.5% to about 5% by weight of the topical skin care composition, and wherein the blend of botanical extracts is present in an amount ranging from about 0.1% to about 1% by weight of the topical skin care composition.

15. The topical skin care composition according to claim 1, wherein the blend of botanical extracts further comprises ginseng extract.

16. The topical skin care composition according to claim 1, wherein the cosmetically acceptable carrier is selected from the group consisting of purified water, oils, alcohols, glycols, and combinations thereof.

17. The topical skin care composition according to claim 1 further comprising additional ingredients selected from the group consisting of penetration enhancers, humectants, lubricants, pharmaceutically active agents, color, fragrance, preservatives, antioxidants, chelators, neutralizers, amino acids, anti-inflammatory agents, anti-cellulite agents, anti-irritants, anti-tack agents, astringents, binders, catalysts, stabilizers, emollients, emulsifiers, surfactants, cell-signaling agents, essential oils, plant/botanical extracts, conditioners, film formers, gelling agents, foaming agents, exfoliants, humectants, vitamins, minerals, pH adjusters, proteins, peptides, neurotransmitters/inhibitors, tactile enhancers, saccharides, solvents and combinations thereof.

18. The topical skin care composition according to claim 1, wherein the topical skin care composition is formulated in a form selected from the group consisting of cream, lotion, serum, facial cleanser, toner, eye cream, sunscreen, stick, spray, filled capsules, impregnated bandage, impregnated personal care device, impregnated towelette, gel, liquid, soap, transdermal patch, powder, liquid powder, cream powder, oil, butter, peel, scrub, mask, elixir, concentrate, capsule, and semi-solid.

19. A method of treating skin comprising the step of:
applying the topical skin care composition according to claim 1 to the skin.

20. A method of treating skin comprising the step of:
applying the topical skin care composition according to claim 15 to the skin.

21. The method according to claim 19, further comprising a step of applying at least one additional skin care composition to the skin,
wherein the at least one additional skin care composition comprises (a) an ascorbic acid source; (b) brown algae extract; and (c) a cosmetically acceptable carrier.

22. The method according to claim 19, wherein the topical skin care composition is applied to skin which suffers from a condition selected from the group consisting of wrinkles, fine lines, hyperpigmentation, uneven tone, loss of firmness, creepiness, surface roughness, dark circles, under-eye puffiness, crow's feet, visible sun damage, redness, dryness, irritation, skin sagging, skin slackening, enlarged pores and combinations thereof.

23. The method according to claim 19, wherein the topical skin care composition is topically applied in an amount and for a period of time sufficient to treat the skin for a condition selected from the group consisting of wrinkles, fine lines, hyperpigmentation, uneven tone, loss of firmness, creepiness, surface roughness, dark circles, under-eye puffiness, crow's feet, visible sun damage, redness, dryness, irritation, skin sagging, skin slackening, enlarged pores and combinations thereof.

23. The method according to claim 19, wherein the topical skin care composition is applied at least once a day.

24. The method according to claim 19, wherein the topical skin care composition is applied twice a day.

25. The method according to claim 19, wherein more than one topical skin care composition is applied.

26. The method according to claim 19 further comprising the step of:
administering an oral supplement formulated to support skin health.

27. The method according to claim 26, wherein the oral supplement is administered at least once a day.

28. A method of treating skin comprising the steps of:
applying a topical skin care composition to the skin of a subject, and
administering to the subject an oral supplement formulated to support skin health,
wherein the topical skin care composition comprises (a) an ascorbic acid source; (b) brown algae extract; (c) a blend of botanical extracts comprising cucumber extract, watercress extract, birch leaf extract, red clover blossom extract, and St. John's wort extract; and (d) a cosmetically acceptable carrier; and
wherein the oral supplement comprises (a) bilberry extract, (b) quercetin, (c) beta-carotene, (d) co-enzyme Q-10, (e) lipoic acid, (f) vitamin A, (g) vitamin B, (h) vitamin C, (i) vitamin D, (j) vitamin E, (k) selenium, (l) zinc, and (m) copper.

29. The method according to claim 28, wherein the blend of botanical extracts further comprises ginseng extract.

30. The method according to claim 28 further comprising a step of:
applying at least one additional skin care composition to the skin of the subject,
wherein the at least one additional skin care composition comprises (a) an ascorbic acid source; (b) brown algae extract; and (c) a cosmetically acceptable carrier.
